(19) United States
(12) Patent Application Publication
(10) Pub. No.: US 2025/0241533 A1
(43) Pub. Date: Jul. 31, 2025

(54) METHODS AND SYSTEMS FOR LINE SCAN ALIGNMENT IN SCANNING LASER OPHTHALMOSCOPY

(71) Applicant: Optos plc, Dunfermline (GB)

(72) Inventor: Daniel Hurst, Dunfermline (GB)

(73) Assignee: Optos plc, Dunfermline (GB)

(21) Appl. No.: 18/428,041

(22) Filed: Jan. 31, 2024

(51) Int. Cl. A61B 3/12; A61B 3/10

(52) U.S. Cl. CPC A61B 3/1225; A61B 3/102

(57) ABSTRACT

Methods and systems for facilitating or improving line scan alignment in a scanning laser ophthalmoscope (SLO) use one or more optical markers located or positioned on a region of an optical element implemented in the SLO device. The optical marker has a predetermined shape and size that assists in detecting any misalignment and/or realigning of the scanned data (such as the line scan) so as to improve the accuracy or integrity of a final scan of a patient's eye.

### FIELD

The present disclosure relates generally to ophthalmic scanning systems and methods. More particularly, the disclosure relates to ophthalmic imaging systems and methods with improved accuracy during scanning.

### BACKGROUND

Ophthalmoscopes can include multiple scanning modalities in the same instrument. For example, ophthalmoscopes can include both a scanning laser ophthalmoscope (SLO) modality and an optical coherence tomography (OCT) modality to capture two-dimensional (2D) and three-dimensional (3D) images of the retina of the eye. The SLO and OCT modalities use separate laser light beams that are directed toward the retina of the eye for capturing images thereof.

When operating the ophthalmoscope in the SLO imaging modality, light passes through both a horizontal scanning mirror and a vertical scanning mirror before and after the eye is scanned in order for the retinal image to be generated. However, if the different scanning mirrors in the SLO (e.g., horizontal and vertical scanning mirrors) are not synchronized or well-aligned with respect to each other, or if the components of the SLO are not aligned and timed properly, the resulting scan that is obtained may include misalignment among the individual pixels or sets of pixels, causing imprecision in the final retinal image obtained from combining such pixels. This may cause a substantial delay in providing the retinal image for the user's review or analysis, therefore causing inconvenience or a possible delay in diagnosing problems in the retina. Additionally, such a misalignment may cause reduction in the quality of the final retinal image that is obtained and/or introduce aberrations into the final retinal image, in which case the retinal image may need to be recaptured, thereby introducing further delays. As such, there is a need for SLO and OCT systems that are designed to address such issues.

### SUMMARY

Disclosed herein are methods and systems for facilitating or improving line scan alignment in a scanning laser ophthalmoscope (SLO) using an optical marker(s) located or positioned on a region of an optical element implemented in the SLO device. The optical marker has a predetermined shape and size that assists in detecting any misalignment and/or realigning of the scanned data (such as the line scan) so as to improve the accuracy or integrity of a final scan of a patient's eye.

According to one example (“Example 1”), a scanning laser ophthalmoscope (SLO) for scanning a retina of an eye includes: a light source emitting a scanning beam, a detector, at least one optical element configured to direct the scanning beam, at least one optical marker disposed at a predetermined region of the at least one optical element, a first scanning element configured to direct the scanning beam between the light source and the at least one optical element, and a second scanning element configured to direct the scanning beam with respect to the at least one optical element. The detector is configured to generate scan data of the retina of the eye comprising a plurality of line scans using the at least one optical element, and a portion of the at least one optical marker is included in each line scan of the plurality of line scans to indicate an alignment of the line scan relative to another line scan of the plurality of line scans.

According to another example (“Example 2”) further to Example 1, the SLO includes a line start sensor disposed in an optical path of the scanning beam and configured to output a signal to the detector to record a new line scan each time the scanning beam is directed to the line start sensor by the first scanning element.

According to another example (“Example 3”) further to Example 2, the detector is operatively coupled with the light source.

According to another example (“Example 4”) further to any one of Examples 1-3, the plurality of line scans are generated by scanning the retina along a first direction using the first scanning element and positions of the plurality of line scans are varied along a second direction orthogonal to the first direction using the second scanning element.

According to another example (“Example 5”) further to any one of Examples 1-4, the SLO includes an optical filter disposed between the detector and the first scanning element and configured to filter the scanning beam to be received by the detector.

According to another example (“Example 6”) further to Example 5, the optical filter is configured to filter a reflected light from the eye and allow a fluorescent light to pass through the optical filter and thereby be received by the detector.

According to another example (“Example 7”) further to Example 6, the SLO includes a beam splitter and a second detector configured to receive the reflected light before being filtered out by the optical filter, thereby obtaining scan data via the second detector that includes one or more portions corresponding to the at least one optical marker.

According to another example (“Example 8”) further to any one of Examples 1-7, the at least one optical marker includes a plurality of optical markers, the plurality of optical markers including: a line-start optical marker defining a first end of the line scans, and a line-end optical marker defining a second end of the line scans.

According to an example (“Example 9”), an ophthalmic imaging system includes the SLO of any one of Examples 1-8. The system includes one or more processing units and one or more memory units storing thereon instructions that, when executed by the one or more processing units, cause the one or more processing units to: receive the scan data generated by the SLO; detect a plurality of optical marker portions in the plurality of line scans of the scan data that each correspond to the optical marker; and generate a final scan of the eye by aligning the plurality of line scans relative to each other based upon the detected optical marker portions.

According to an example (“Example 10”), an ophthalmic imaging system includes the SLO of any one of Examples 1-8. The system includes one or more processing units and one or more memory units storing thereon instructions that, when executed by the one or more processing units, cause the one or more processing units to: receive the scan data generated by the SLO, the scan data including an optical marker portion corresponding to the optical marker in a first line scan of the scan data; and align the first line scan relative to a second line scan of the scan data based upon the detected optical marker portion.

According to another example (“Example 11”) further to Example 10, the first line scan is aligned relative to the second line scan at or near real-time, and the second line scan is from the scan data that is previously generated by the SLO.

According to another example (“Example 12”) further to any one of Examples 9-11, the system includes a module for optical coherence tomography (OCT) imaging including optics for OCT imaging that are operatively coupled with the SLO.

According to another example (“Example 13”) further to Example 12, the optics for OCT are coupled with the SLO at a location between the light source and the first scanning element.

According to another example (“Example 14”) further to Example 12, the optics for OCT are coupled with the SLO at a location between the first scanning element and the at least one optical element.

According to another example (“Example 15”) further to Example 12, the optics for OCT are coupled with the SLO at the second scanning element.

According to another example (“Example 16”) further to any one of Examples 12-15, the SLO, the optics for OCT, and at least one of the one or more processing units are enclosed within a common housing.

According to an example (“Example 17”), a method for scanning a retina of an eye using a scanning laser ophthalmoscope (SLO) includes: emitting, by a light source, a scanning beam toward a first scanning element; directing, by the first scanning element, the scanning beam between the light source and at least one optical element, the at least one optical element comprising at least one optical marker disposed at a predetermined region of the at least one optical element; and generating, by a detector, scan data for the retina comprising a plurality of line scans captured via the second scanning element and a second scanning element using the at least one optical element. A portion of the at least one optical marker is included in each line scan of the plurality of line scans to indicate an alignment of the line scan relative to another line scan of the plurality of line scans.

According to another example (“Example 18”) further to Example 17, the method includes: filtering, by an optical filter disposed between the detector and the first scanning element prior to generating the scan data, the scanning beam received by the detector.

According to another example (“Example 19”) further to Example 18, the optical filter is configured to filter a reflected light from the eye and allow a fluorescent light to pass through the optical filter and thereby be received by the detector.

According to another example (“Example 20”) further to Example 19, the method includes: receiving, by a second detector via a beam splitter, the reflected light before being filtered out by the optical filter, thereby obtaining scan data via the second detector that includes one or more portions corresponding to the optical marker.

According to another example (“Example 21”) further to any one of Examples 17-20, the directing the scanning beam between the light source and the at least one optical element includes: directing, by the first scanning element, the scanning beam over a first optical marker of the at least one optical marker that is disposed at a first predetermined region of the at least one optical element; and directing, by the first scanning element, the scanning beam over a second optical marker of the at least one optical marker that is disposed at a second predetermined region of the at least one optical element that is different from the first predetermined region.

According to another example (“Example 22”) further to any one of Examples 17-21, the method includes: detecting a plurality of optical marker portions in the plurality of line scans of the scan data that each correspond to the optical marker; and generating a final scan of the eye by aligning the plurality of line scans relative to each other based upon the detected optical marker portions.

According to another example (“Example 23”) further to any one of Examples 17-21, the method includes: aligning a first line scan of the scan data relative to a second line scan of the scan data based upon an optical marker portion included in the scan data, the optical marker portion corresponding to the at least one optical marker.

According to another example (“Example 24”) further to any one of Examples 17-23, the method includes: performing optical coherence tomography (OCT) in parallel with operating the SLO, such that an output of the SLO is used for tracking movement of the eye in performing the OCT.

According to an example (“Example 25”), a method of generating a scan of an retina of an eye includes: receiving scan data from a scanning laser ophthalmoscope (SLO), the scan data comprising a plurality of line scans that each have an optical marker portion corresponding to an optical marker of at least one optical element of the SLO, such that the scan data includes a plurality of optical marker portions that each indicate an alignment of a line scan relative to another line scan of the plurality of line scans; and aligning each line scan of the plurality of line scans based upon the optical marker portions.

According to another example (“Example 26”) further to Example 25, the method includes: analyzing, in response to receiving the scan data from the SLO, the scan data to detect the plurality of optical marker portions corresponding to the optical marker of the at least one optical element.

According to another example (“Example 27”) further to Example 26, the optical marker is a first optical marker, and the plurality of optical marker portions is a plurality of first optical marker portions. The method includes: analyzing the scan data to detect a plurality of second optical marker portions corresponding to a second optical marker of the at least one optical element; and analyzing the scan data further comprises scaling each line scan based on the plurality of second optical marker portions.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 is a block diagram schematically illustrating an example of an ophthalmic imaging system according to embodiments disclosed herein.

FIG. 2 is an optical schematic showing an example of a scanning laser ophthalmoscope (SLO) according to embodiments disclosed herein.

FIG. 3 is a schematic diagram showing an example of a slit mirror implemented in an SLO according to embodiments disclosed herein.

FIG. 4 is a schematic diagram showing an example of an SLO according to embodiments disclosed herein.

FIG. 5 is a schematic diagram showing an example of possible implementation of optical coherence tomography (OCT) with the SLO of FIG. 4 in a combined SLO and OCT wide-field imaging system, in which the OCT and SLO sources are combined at various parts of the system, according to embodiments disclosed herein.

FIG. 6 is a schematic diagram showing an example of line scans obtained using the SLO according to embodiments disclosed herein.

FIG. 7 is a schematic diagram showing an example of the line scans of FIG. 6 as realigned using the optical marker according to embodiments disclosed herein.

FIG. 8 is a flow chart of a process of operating the ophthalmic imaging system according to embodiments disclosed herein.

FIG. 9 is a flow chart of a process of operating the ophthalmic imaging system according to embodiments disclosed herein.

FIG. 10 is an optical schematic showing another example of an SLO according to embodiments disclosed herein.

FIG. 11 is a block diagram of example physical components of a computing device.

It should be understood that some of the drawings and replicas of the photographs may not necessarily be shown to scale, unless otherwise indicated. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### Description of Various Embodiments

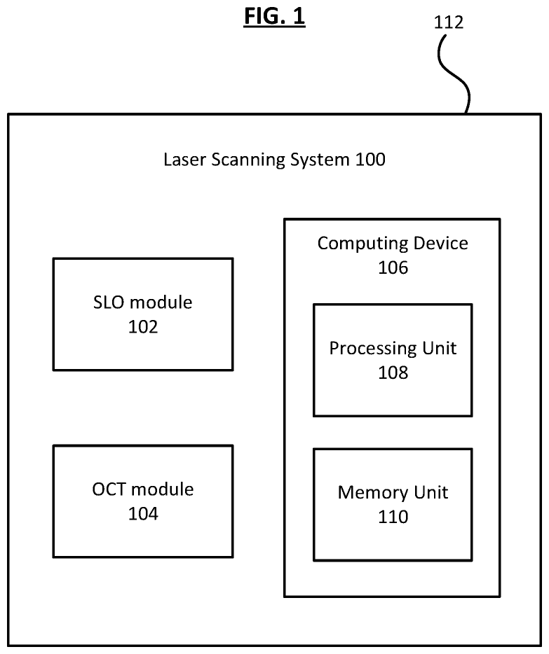

FIG. 1 is a schematic diagram illustrating an example of an ophthalmic imaging system 100 which includes a first imaging modality such as scanning laser ophthalmoscopy (SLO) 102 and a computing device 106. In some examples, the system 100 may also include a second imaging modality such as optical coherence tomography (OCT) 104. The components of the ophthalmic imaging system 100 including the SLO module 102, the OCT module 104, and the computing device 106 may be housed inside a housing 112 of a single instrument such as an ophthalmoscope, according to some examples. In some examples, the SLO 102 and the OCT 104 modules may be housed inside the housing 112 with the computing device 106 located external to the housing 112 or within a different housing. In some examples, any of the components may be located in a different housing from the rest of the components. As an example, computing device 106 may be remote from the modules of SLO 102 and OCT 104.

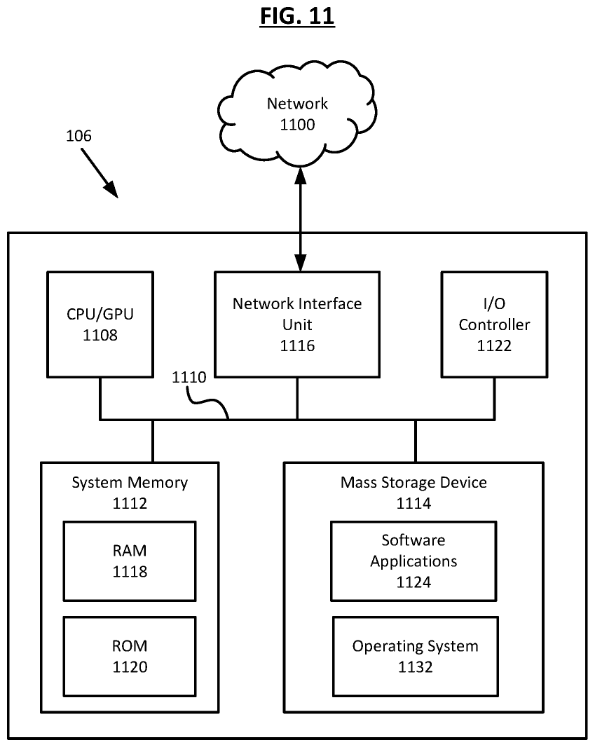

The computing device 106 operates to control the SLO 102 and OCT 104 modules and has at least one processing unit 108 such as a central processing unit (CPU) and/or graphics processing unit (GPU), and a memory unit 110 that stores instructions that, when executed by the at least one processing unit 108, causes the processing unit 108 to perform one or more methods and functions described herein. The computing device 106 will be described in more detail below in a description of FIG. 11.

In some examples, the SLO module 102 uses confocal laser scanning microscopy for diagnostic imaging of the retina of the eye. The imaging may be two-dimensional (2D) imaging. The SLO module 102 may use a laser light beam to scan across the retina in a raster pattern to illuminate successive elements of the retina, point-by-point. The light reflected from each retinal point may be captured by a photomultiplier. The output of the photomultiplier may be recorded and displayed in a digital format. In this manner, the SLO module 102 is able to produce high-contrast, detailed images of the retina.

In some examples, the process of operating the OCT module 104 is noninvasive and uses light waves to obtain high resolution cross-sectional images of the retina. The cross-sectional images may be generated by analyzing a time delay and magnitude change of low coherence light as it is backscattered by ocular tissues. Layers within the retina can be differentiated and retinal thickness can be measured to aid in the early detection and diagnosis of retinal diseases and conditions including, by example, glaucoma, age-related macular degeneration (AMD), and diabetic eye diseases including but not limited to diabetic retinopathy (DR).

In some examples, images are captured sequentially by the first imaging modality such as the SLO 102 and the second imaging modality such as the OCT 104, such that a first image is captured by the first imaging modality (SLO 102), and thereafter the first image is used a reference for capturing a second image by the second imaging modality (OCT 104). In some examples, the images are captured simultaneously or in parallel using both the SLO 102 and OCT 104 modules to obtain both the SLO and OCT outputs, such that the SLO output may be used for tracking any movement of the eye while the OCT output is being generated, since the OCT output generally takes a longer time to generate than the SLO output. In some examples, the ophthalmic imaging system 100 may include suitable components for SLO and OCT imaging that are arranged in configurations as explained in U.S. Pub. No. 2021/0100450 (Optos PLC), the disclosure of which is incorporated herein by reference in its entirety.

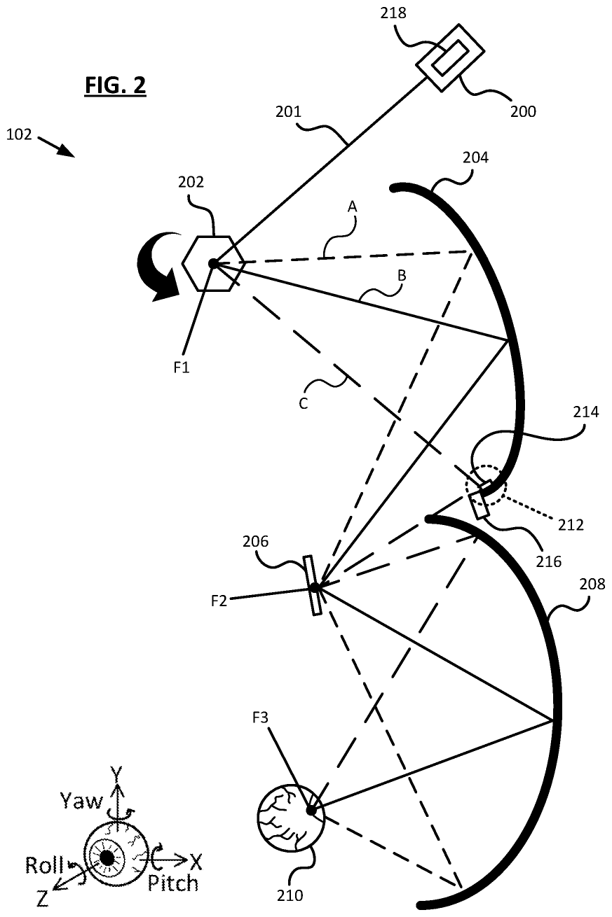

Referring to FIG. 2, a schematic view of the ophthalmic imaging system 100 is shown, in which the SLO module 102 includes a light source 200 emitting beam of light, that is, a scanning beam 201, and a plurality of scan relay elements. The scan relay elements include a first scanning element 202, a second scanning element 206, and at least one optical element that is positioned and configured to direct the scanning beam 201. In some examples, the at least one optical element may include a first optical element 204 which may be a scan compensation element such as a slit mirror, and a second optical element 208 which may be a scan transfer element such as a main mirror. The optical elements 204 and 208 are positioned and configured to direct the scanning beam 201. The first scanning element 202 may be or include a rotating polygon scanning mirror, and the second scanning element 206 may be or include an oscillating plane scanning mirror or a planar scanning mirror coupled to a galvanometer motor. The first optical element 204 may be an ellipsoidal mirror. The second optical element 208 may be an aspherical mirror. It is to be appreciated that the first and second optical elements may have an alternative form. The scanning elements 202 and 206 may be referred to as a scanning device or a plurality of individual scanning devices. It is to be understood that the SLO module 102 illustrated in FIG. 2 are just an example of a configuration that can be used with the embodiments described herein, and that additional configurations are contemplated.

The optical element 204 includes an optical marker 214 positioned thereon. In some examples, the optical marker 214 may be positioned at or near a periphery region 212 or end region of the optical element 204, or at any other suitable location on the optical element 204. There may be one marker or multiple markers positioned on the optical element 204. In some examples, a line start sensor 216 is disposed in or along an optical path of the scanning beam 201, and may be operatively coupled with the optical element 204. In some examples, the line start sensor 216 may be positioned proximate to the optical element 204, for example at/near an edge or near the periphery region 212 of the optical element 204. The line start sensor 216 may be configured to generate and output a signal (a line-start pulse signal) to a detector 218 to start recording the scan data when the scanning beam 201 is directed to the line start sensor 216 by the first scanning element 202. For example, the line-start pulse signal may be generated when the scanning beam 201 crosses an optical fiber in the line start sensor 216. In some examples, the detector 218 may be a separate component or device that is operatively coupled with the light source 200 such that the detector 218 and the light source 200 may be positioned at the same location or adjacent to one another. In some examples, the light source 200 may be implemented with the detector 218 as shown in FIG. 2 as a single device, or devices within a common housing, such that in addition to emitting the scanning beam 201, the device may also be capable of detecting or receiving light, such as the light reflected back from the first scanning element 202 or from the optical element 204.

If the system 100 is implementing only the SLO module 102 (and thus omitting the OCT module 104) to obtain SLO images, the scanning beam 201 may be a laser of suitable wavelength as used in SLO applications. If the system 100 is implementing both the SLO 102 and the OCT 104 modules to obtain SLO and OCT images, the scanning beam 201 may be a collimated light which includes the laser for SLO applications and a superluminescent diode (SLD) for OCT applications, for example. It should be appreciated that any suitable source of collimated light may be used, such as a single frequency laser diode, vertical-cavity surface-emitting laser, wavelength swept laser source, pulsed laser source, or other source that has enough intensity and to be well collimated and produce adequate retinal illumination. In OCT applications (e.g., for OCT module 104), the SLD may be used due to the short coherence lengths required to discriminate the retinal layers from the resultant interferometric data. The SLD may be free space or fiber coupled into standard or polarization maintaining fiber to the scan system. A swept source laser may also be used in OCT applications, whereby the wavelength of the source is tuned over a given range.

In some examples, one or more of the scanning elements may include one or more of: an oscillating plane mirror, a galvanometer mirror, a MEMS mirror, a rotating mirror, prism or polygon scanner, and/or a resonant mirror, for example. It should be appreciated that other suitable scanning elements may be used, such as line scanning produced with a laser line source, or equivalent. Line scanning may be used as an effective alternative to point scanning. A line source may produce a line illumination on the retina which is scanned orthogonally by a slow scanner. The line illumination may be detected by a linear pixel array, and a 2D image may be built up by rotating the slow scanner.

In some examples, each of the first scanning element 202 or the second scanning element 206 may be a single element or an arrangement of two or more elements as suitable to provide a scan at a respective focal point F1 or F2, as shown, at which the scanning element is disposed. The focal points F1 and F2 are the foci of the optical element 204, and the focal points F2 and F3 are the foci of the optical element 208. The first scanning element 202 is positioned at focal point F1, the second scanning element 206 is positioned at focal point F2, and the eye 210 is positioned at focal point F3 (also referred to as a virtual scanning point). The resulting scan may be a 2D scan or scan pattern of the scanning beam 201 as the light sweeps through the virtual scanning point (e.g., through F3) inside the eye 210.

In some examples, the first scanning element 202 provides either a vertical, horizontal, or patterned scan which is incident on the optical element 204, to a point on the second scanning element 206 via the optical element 204. The scans may be one-dimensional (1D) or two-dimensional (2D) light scans, for example. The axes of the first scanning element 202 and the second scanning element 206 may be arranged to create a 2D light scan, such as in the form of a raster scan pattern of the scanning beam 201. The alignment of the first and second scanning elements 202, 206 may be orthogonal, substantially orthogonal, or arranged to generate an arbitrary scan geometry about the optical elements 204 and 208.

In some examples, the second scanning element 206 provides a plurality of scans, for example 1D or 2D light scans, which may comprise horizontal scans, vertical scans, or arbitrary patterns of the scanning beam 201. The scans provided by the first scanning element 202 and the scans provided by the second scanning element 206 are different from each other, for example with respect to the orientation of the scans. In some examples, one of the scanning elements may provide vertical scans of the retina, and the other scanning element may provide horizontal scans of the retina. The scanning beam 201 is directed towards a patient's eye 210 via the scanning elements 202 and 206, and the optical elements 204 and 208, such that an ultra-wide field scan angle is achieved at the pupil plane of the eye 210. A “wide field” scanning refers to a scan angle in excess of 50 degrees in one or two dimensions. An “ultra-wide field” scanning refers to a scan covering substantially the entire retina of the eye 210. In some examples, the plurality of line scans may be generated by scanning the retina along a first direction using the first scanning element 202, and positions of the plurality of line scans are varied along a second direction using the second scanning element 206, where the second direction is orthogonal to the first direction.

As illustrated in FIG. 2, the path of the scanning beam 201 is shown in a 1D scan produced by one oscillation or rotation (shown by the curved arrow) of the first scanning element 202. Path “A” is an example of the scanning beam 201 reflected from the first scanning element 202 at the start of the rotation. Path “B” is an example of the scanning beam 201 reflected from the first scanning element 202 at an intermediate point of the rotation. Path “C” is an example of the scanning beam 201 reflected from the first scanning element 202 at the end of the rotation.

The components of the SLO module 102 may be arranged such that the rotational axis of the first scanning element 202 is substantially parallel to a line joining the two foci of the optical element 208 (i.e., F2 and F3) such that the scanning beam 201 is scanned across the secondary axis of the optical element 204. Furthermore, the first scanning element 202 may produce a 1D or 2D scan which is incident on the optical element 204. The optical element 204 may also therefore produce a 1D or 2D scan. The components of the SLO module 102 may be arranged such that the line joining the two foci of the optical element 208 (i.e., F2 and F3) lies substantially on a plane defined by the scan (e.g., 1D vertical scan) produced by the optical element 204.

The first and second scanning elements 202 and 206 may thus together create a light scan, for example a 2D scan, in the form of a raster scan pattern from a single point in space at or near the focal point F3 at or in the eye 210 of the patient. The first and second scanning elements may have operating parameters which include the amplitude of the oscillation and the rotational offset of the oscillation. The operating parameters also include the velocity of oscillation. Both of these operating parameters may be selected to control the direction and pattern of the light scan from the apparent point source. In some examples, the first and second scanning elements may be housed in a rotation mount (not shown) that can adjust the centering (or eccentricity) of the scanning beam 201 on the retina of the eye 210, which provides the ability to “move” the imaging field across the retina.

In some examples, the optical element 208 directs the scanning beam 201 to the patient's eye 210 and may be configured to provide a field of view with a predetermined angle, for example a 200-degree field of view (external angle), in both the vertical and horizontal directions (e.g., 200 degrees×200 degrees) on the retina. It should be appreciated that the optical element(s) may also be configured to provide at a substantially lesser or greater field of view in one or both of the horizontal and vertical directions. For example, the horizontal direction may be in the X-axis with respect to the eye 210, and the vertical direction may be in the Y-axis with respect to the eye 210.

In some examples, it should be appreciated that the scan relay elements may take other forms. For example, the scan relay elements may include an elliptical mirror, a pair of parabolic mirrors, a pair of paraboloidal mirrors, or a combination of any of these components. The common technical feature provided by any of these component arrangements is that the scan relay element includes two foci and produces a light scan, for example a 1D light scan.

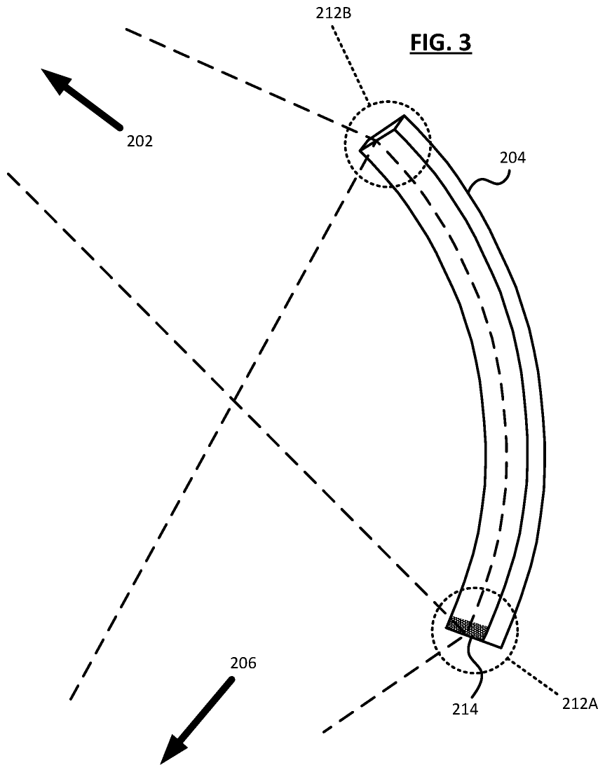

FIG. 3 illustrates an example of the optical element 204 showing a position of the optical marker 214. The marker 214 may be positioned at any suitable location on the surface of the optical element 204 such that each scan performed or generated by the first scanning element 202 will include a portion of the optical marker 214. In some examples, the optical marker 214 may cover a portion or the entirety of the periphery region 212 of the optical element 204 such that the optical marker 214 is more visible in each scan. The broken lines show the range of optical path (e.g., paths “A”, “B”, and “C”) that may be taken between the first scanning element 202 and the second scanning element 206. The marker 214 may be detectable via the first scanning element 202 and/or the second scanning element 206, for example by the detector 218.

In some examples, the optical marker 214 may extend only along one side of the periphery region 212 (shown as region 212A) while the other sides of the periphery region 212 (for example, region 212B) may remain free of the optical marker 214. In some examples, both regions 212A and 212B may have an optical marker 214 located thereat. In examples, the use of multiple markers further enables scaling of a line scan (in addition to alignment, as would be possible with only one optical marker), such that variability in scan time for a given line scan may be accounted for when combining line scans into a resulting scan. In some examples, if there are multiple optical markers 214 located at different regions on the optical element 204, each optical marker may have a color or pattern that is unique to the optical marker, so as to be visually distinguishable from other markers. In some examples, the multiple optical markers 214 may include a line-start optical marker defining a first end of the line scans, and a line-end optical marker defining a second end of the line scans, where the line-start and line-end optical markers may be used to compensate not only a line-start position of the scan but also variations in the speed of the scan caused by the variation in the rotation speed of a first scanning element, which may be or include a rotating polygon scanning mirror. In some examples, the optical marker may be applied via a colored paint or coating. In some examples, the optical marker may be applied via an adhesive such as a sticker. As another example, the optical marker may be etched or otherwise formed in the optical element 204 itself.

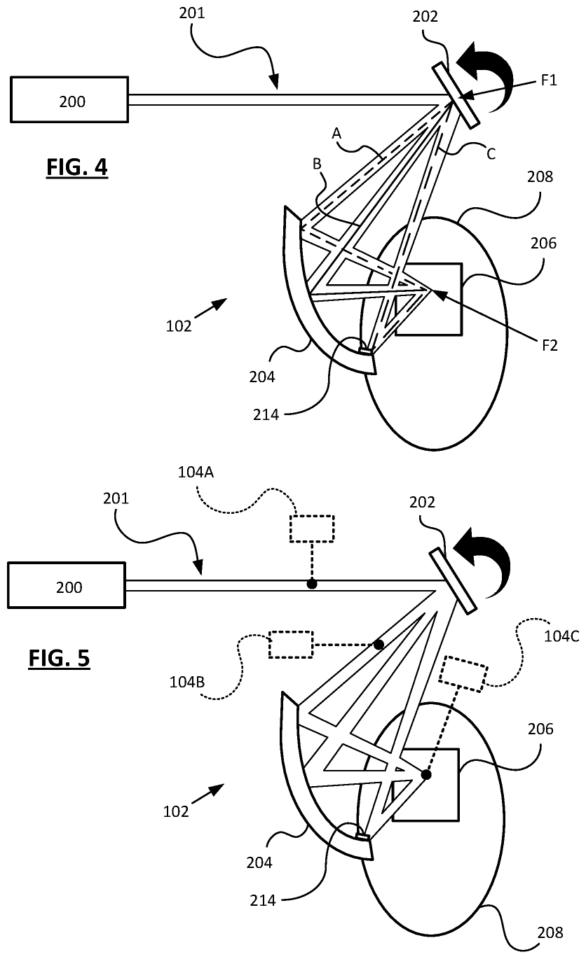

FIG. 4 shows an example of how the components of the SLO module 102 may be positioned relative to each other. The paths “A”, “B”, and “C” are shown to illustrate the paths taken by the scanning beam 201 generated by the light source 200 between the first focal point F1 on the first scanning element 202 and the second focal point F2 on the second scanning element 206 as reflected by the optical element 204. The first scanning element 202 may be rotatable with respect to the focal point F1. The optical marker 214 is positioned so as to be detectable in the light scans (1D or 2D) obtained via the first scanning element 202 and/or the second scanning 206, for example by the detector 218. Although not shown, the eye 210 is disposed at the focal point F3 (not shown) of the optical element 208, where the focal points F2 and F3 are the foci of the optical element 208. The second scanning element 206 may be rotatable with respect to the focal point F2.

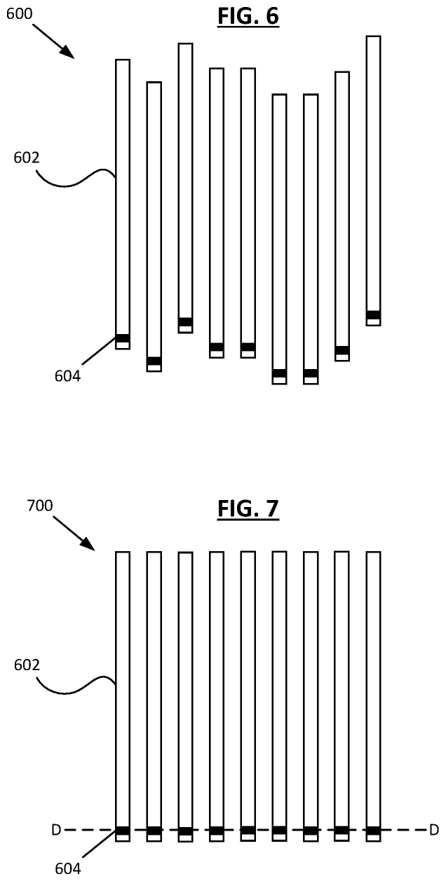

FIG. 5 illustrates some of the different locations in which the OCT optics 104A, 104B, and 104C may be disposed or coupled to the SLO module 102 in a combined OCT/SLO system, such as the system 100 shown in FIG. 1.

In a first example, the OCT optics 104A are operatively coupled with the SLO module 102 at a location along the scanning beam 201 between the light source 200 and the first scanning element 202, such that the OCT and SLO beams are combined (as the collimated scanning beam 201) before the first scanning element 202. In this arrangement, the SLO illumination source or light source 200, the first scanning element 202, the optical elements 204 and 208, and the second scanning element 206 are provided as before. The illuminator or light source 200 may emit a laser beam as the scanning beam 201. The OCT optics 104A provide a collimated beam from a fiber-delivered OCT source via the OCT interferometer such that the emitted beam from the OCT optics 104A forms the OCT sample beam. The OCT optics 104A may also contain local scanning optics such that an OCT scan point can be relayed through the scan relay and scan transfer means to the patient's retina.

The illumination source used for the OCT optics 104A may in one example comprise the SLD which may for example operate over any region of the NIR-IR spectrum. Alternatively, the illumination source used for the OCT optics 104A may be a swept laser source or a pulsed laser source. In this configuration, the OCT scan system may be propagated to the optical elements 204 and 208 via the first scanning element 202. The optical system in the OCT scan system may propagate the OCT illumination such that the apparent point source is co-located at the first scanning element 202. The OCT illumination can then be directed to the entirety of the retina which is addressable by the combination of scanning elements 202 and 206, or a sub-section of the retina by fixed angle settings of the scanning elements 202 and 206. Moreover, the subsection of the addressed retina may then be imaged via the integrated scan means in the OCT scan system, thereby providing utility for wide-field images (e.g., 2D and/or 3D) or targeted subsections (e.g., 2D and/or 3D) of the retina.

In a second example, the OCT optics 104B are operatively coupled with the SLO module 102 at a location between the first scanning element 202 and the optical element 204, such that the OCT and SLO beams are combined after the first scanning element 202 but before the second scanning element 206. In this configuration, the OCT illumination is directed to the second scanning element 206 via the optical element 204 and an additional optical element (not shown), which may for example be a beam splitter.

The optical layout for the second example is such that the apparent point source is located at the second scanning element 206, corresponding to one focus of the optical element 208. The OCT illumination can then be directed to the entirety of the retina addressable by rotation of second scanning element 206, rotation of an optical element within the OCT optics 104B, or rotation of the OCT optical assembly. In addition, the OCT illumination can be directed to a sub-section of the retina by fixed angle settings of the second scanning element 206 and integrated OCT scan system within the OCT optics 104B. Moreover, the subsection of the addressed retina can then be imaged via the integrated scan means in the OCT scan system, thereby providing utility for wide-field retinal images (e.g., 2D and/or 3D) or targeted subsections (e.g., 2D and/or 3D) of the retina.

In a third example, the OCT optics 104C are operatively coupled with the SLO module 102 at the second scanning element 206, such that the OCT and SLO beams are combined directly on the second scanning element 206. In this configuration, the SLO and OCT images do not have to be on the same point on the retina. The OCT illumination is directly coupled to the second scanning element 206 such that the apparent point source is located at the second scanning element 206, corresponding to one focus of the optical element 208. This path is independent of the optical element 204 or the first scanning element 202.

The OCT illumination can then be directed to the entirety of the retina addressable by rotation of the second scanning element 206, rotation of an optical element within the OCT optics 104C, or rotation of the OCT optical assembly. In addition, the OCT illumination can be directed to a sub-section of the retina by fixed angle settings of the second scanning element 206 and integrated OCT scan system within the OCT optics 104C. Moreover, the subsection of the addressed retina can then be imaged via the integrated scan means in the OCT scan system, thereby providing utility for wide-field retinal images (e.g., 2D and/or 3D) or targeted subsections (e.g., 2D and/or 3D) of the retina.

In each of the above configurations as explained in the first, second, and third examples, the OCT illumination may be scanned across or directed to a portion of the elliptical section of either or both the optical element(s) 204 and/or 208, such that the OCT illumination is reflected from at least one non-spherical optical element, and in some embodiments from at least two. The OCT system may be configured and operated according to the embodiments as explained in U.S. Pub. No. 2015/0216408 (Optos PLC), the disclosure of which is incorporated herein by reference in its entirety.

FIG. 6 shows scan data 600 as obtained using the first scanning element 202 and/or the second scanning element 206 according to embodiments disclosed herein. The scan data 600 includes a plurality of line scans 602 (e.g., 1D vertical scans) each generated separately as the light sweeps through the virtual scanning point inside the patient's eye. In order to perform the SLO scanning, the first scanning element 202, for example a polygon scanning mirror, must be in the correct location and position. In order to confirm that the scanning element is in its correct location and position, the line start sensor 216 is implemented such that when the scanning beam 201 (e.g., laser beam) scans over the sensor 216, a signal (a line-start pulse signal) is generated and transmitted to the detector 218 (e.g., a frame grabber). Such line-start pulse signal may be subjected to positional misalignment (hereinafter referred to as “positioning jitter”) with respect to the position of the first scanning element 202. Various factors may cause or affect the severity of the positioning jitter, including but not limited to: tolerances in the system 100; alignment of the sensor 216; differences in the polygon facets of the polygon scanning mirror implemented as the first scanning element 202; changes in a shape of the spot on the sensor 216 depending upon where the sensor 216 is fitted; and/or the wavelength of the light used (e.g., due to the different imaging modes being implemented), among others. The positioning jitter resulting from such factors may cause image artefacts where the lines in the resulting image data are not well-aligned with respect to each other. Such image artefacts may impact manufacturing yield as well as cause image quality issues for the end user, such as the patient or the patient's doctor.

As shown in FIG. 6, the positioning jitter causes optical marker portions 604 that are included in the individual line scans 602 to be in a staggered or misaligned configuration with respect to each other. Each of the optical marker portions 604 is a portion of the optical marker 214 that is scanned by the suitable scanning element and captured as part of each of the line scans 602. In FIG. 7, the positioning jitter is reduced or eliminated when the line scans 602 are aligned based upon the optical marker portions 604, such as by positioning the optical marker portions 604 along a common line D-D. Doing so allows the remaining portions of the line scans 602 to also be repositioned and aligned with respect to each other, causing a final scan 700 which is generated by combining all the repositioned line scans 602 into a single scan data, such as a single scan image, for example 2D or 3D scan data as suitable. As noted above, similar techniques may be used in instances where multiple optical markers are used, such that each line scan comprises multiple optical marker portions that are each aligned according to a respective line (e.g., along D-D and another common line, not pictured). Such aspects may further enable scaling of each line scan, thereby accounting for variability in scan time for each respective line scan.

As explained above, the optical marker(s) 214 may be added to the optical element 204 in the optical path of the scanning beam 201 in any suitable position or location. Examples of such location may be at the peripheral region 212 of the optical element 204, near the peripheral region 212, or within any other region or area of the optical element 204 that is located outside of the imaging field of view, which may be predetermined by the system 100. The optical marker 214 may be one or more lines drawn or added into the optical element 204, a block of colored region (e.g., a black region of a polygonal shape) that is drawn or added on a surface of the optical element 204, a bicolor (e.g., black-and-white) bar code drawn or added onto the surface of the optical element 204, or any other suitable shape or configuration that may be contemplated. The optical maker 214 is to be an easily identifiable mark or visual indicator that is captured in each of the plurality of line scans 602 that is used to align and ultimately form the final scan 700. It will be appreciated that final scan 700 is illustrated as including optical marker portions 604 for illustrative purposes and, in other examples, a final scan may omit such optical marker portions after alignment (e.g., such that they are not displayed in a final scan that is provided to a user).

According to some examples, the first scanning element 202, as explained above, may be or include a polygon scanning mirror, and the scanning element 202 may be arranged to firstly scan the light over the line start sensor 216, and in response, the line start sensor 216 generates and transmits the line-start pulse signal to the detector 218 (frame grabber) to indicate that the scanning element 202 is in the correct location and position to start capturing the image data. However, as explained above, the line start sensor 216 may be prone to introduce positioning jitter into the scan. Thus, the line start sensor 216 may be used as a “macro” indicator of the position of the scanning element 202. On the other hand, the optical marker(s) 214 may be used as a “micro” indicator for the position of the scanning element 202 and/or any possible misalignment or positioning jitter caused by the line start sensor 216, when the scanning element 202 scans the line of light over the optical marker 214 on the optical element 204. Because the optical marker 214 is in a fixed position, it may thus be used to more accurately align the adjacent line scans 602 relative to each other before being combined to form the final scan 700 as explained above.

In some examples, once the line scans 602 are scanned over the full field of view of the imaging device (that is, the SLO module 102 or apparatus), the scan data 600 that is obtained is post-processed such that the adjacent line scans 602 of the scan data 600 are aligned with respect to each other, based upon the detected optical marker portions 604 in the line scans 602, to remove the positioning jitter from the data of the final scan 700. Aligning the adjacent line scans may include, for example, detecting the position of the optical marker portion 604 in each line scan 602 and then aligning the optical marker portion 604 in each line with the position of the optical marker portion 604 of the adjacent line(s), or aligning the optical marker portion 604 in each line with a common line D-D. Beneficially, aligning the optical marker portions 604 removes image artefacts associated with the positioning jitter (e.g., as may be introduced by the line start sensor 216 and/or variability of other components of the ophthalmic imaging system), thereby generating a higher-quality ophthalmic image (SLO image). As noted above, the portion of the final scan 700 containing the optical marker 214 or optical marker portions 604 may be cropped so as to not have such marker be displayed to the end user. In some examples, the aligning of the line scans may be performed at or near real-time, as new or additional line scans are obtained or generated. For example, whenever a new line scan is generated, the new line scan may be automatically aligned with the previously generated line scan (that is, the line scan that was last generated prior to generating the current, new line scan) based upon the optical marker portions that are included in the new line scan and the last generated line scan. Therefore, instead of in the post-processing, the adjustment or alignment of the line scans may be performed “on the fly” as each new line scan is generated, without having to wait for a predetermined number of line scans to be generated.

In some examples, the SLO module 102 may not include the line start sensor 216 and instead uses the optical marker(s) 214, independently from the line start sensor 216, to determine the position of the first scanning element 202. In the examples implementing the line start sensor 216, the scan data may be recorded continuously after the line start sensor 216 transmits the line-start pulse signal, such that any suitable scan data processing technique may be used to detect the presence and location of the optical marker 214 in the acquired line data. In examples where the line start sensor 216 is omitted, the start of a new line scan may be identified based on identifying the optical marker(s) 214 in the scan data, such as by detecting the presence of the optical marker portions 604 in the line scans 602.

In some examples, the line start sensor 216 may beneficially reduce the likelihood of possible errors in the analysis by providing an indication of the general area or region in which the optical marker 214 is likely to be located or is roughly expected to be found, thereby reducing the likelihood of the scan data processing techniques erroneously determining that an anomaly that is present in the patient's eye is the optical marker 214. Such erroneous determination or confusion may occur if the anomaly resembles the shape and/or size of the optical marker 214 that is implemented. As such, in some examples, the optical marker 214 may be configured or designed to avoid such error or confusion of being possibly mistaken for any such anomaly in the eye, by forming a more complex shape or pattern that is unlikely to occur naturally in the eye. Examples of such complex shape or pattern may include, but are not limited to, bicolor bar codes or a string of alphabetical letters and/or numbers, among others. As another example, such an anomaly may be filtered out of the scan data (e.g., such that it is not identified as an optical marker portion) based on evaluating a period at which optical marker portions are present in the scan data and determining whether a subsequent feature of the scan data is likely to be another optical marker portion or an anomaly depending on if the feature occurs at or near an expected period for the optical marker portions. Such evaluation and/or determination may be performed by software processing, for example.

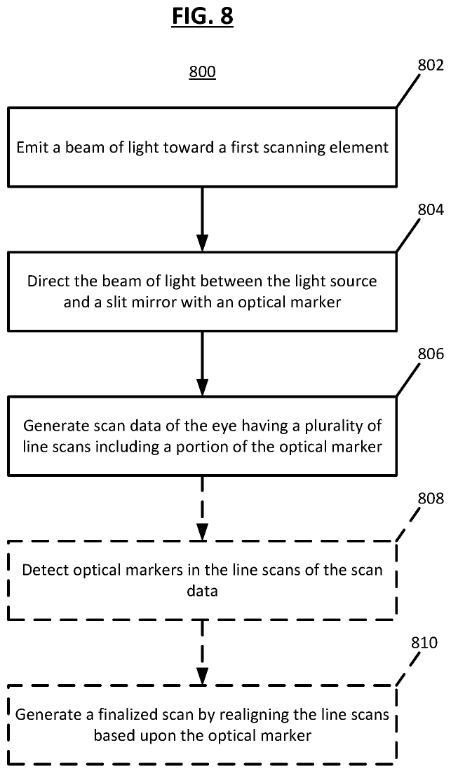

FIG. 8 shows a method 800 of scanning a retina of an eye using the SLO, and generating line scans with the optical marker for SLO imaging, according to examples disclosed herein. Each of the blocks in the method 800 represents a step or operation, which may be performed by one or more devices or components as further explained.

In step 802, the light source emits a scanning beam toward the first scanning element. In step 804, the first scanning element directs the scanning beam between the light source and the optical element. The optical element includes an optical marker(s) disposed at a predetermined region of the optical element. It is to be understood that the scanning beam may be also directed toward the optical element via the second scanning element and then reflected from the optical element via the first and second scanning elements disposed therebetween, to be detected, for example by a detector. According to some examples, the optical element may be a mirror of any suitable size and/or shape. In some examples, the scanning beam may be directed over a first optical marker that is disposed at a first predetermined region of the optical element, and be directed over a second optical marker that is disposed at a second predetermined region of the optical element that is different from the first predetermined region. In some examples, the first optical marker may be a line-start optical marker defining a first end of the line scans, and the second optical marker may be a line-end optical marker defining a second end of the line scans. In some examples, the line-start and line-end optical markers may be used to compensate the line-start position of the scan as well as variations in the speed of the scan caused by the variation in the rotation speed of a first scanning element, which may be or include a rotating polygon scanning mirror.

In step 806, scan data for the retina is generated (e.g., by a detector) via the first scanning element and the second scanning element. As explained above, the scan data may be generated in response to the detector detecting the scanning beam that is reflected from the optical element(s) via the first and second scanning elements. The scan data includes a plurality of line scans, and a portion of the optical marker is included in each line scan, thereby indicating an alignment of a given line scan relative to one or more other line scans (e.g., one or more adjacent line scans) of the plurality of line scans.

In some examples, at step 804, after step 804, or before step 806, the light may also be scanned over a line start sensor in addition to the optical marker(s). Thereafter, the line start sensor may output a signal (line-start pulse signal) to the detector (frame grabber). The signal may cause the detector to start recording the scan data (or image data) or to record a new line scan that forms the scan data. The detector may record a new line scan each time the line-start pulse signal is detected. The signal may additionally or alternatively cause the detector to segment the scan data (or image data) that is recorded into a plurality of line scans. In some examples, the aforementioned steps may be repeated for the horizontal positions in the eye by scanning a line in a first direction (e.g., vertical) using the first scanning element, which may be a polygon scanning mirror, and varying the position in a second direction orthogonal to the first direction (e.g., horizontal) using the second scanning element, which may be a galvanometer mirror, for example.

Steps 808 and/or 810, shown in broken lines, may be performed by any one or more suitable device or components, including but not limited to the SLO apparatus, the computing device(s), the processing unit(s) of the computing device, an external or auxiliary computing device which may be physically attached to and operatively coupled with the main computing device or the SLO apparatus, a remote computing device which may be remotely but operatively coupled with the main computing device or the SLO apparatus, etc., as suitable. In step 808, instances of the optical marker(s) are detected as optical marker portions within the line scans of the scan data. In step 810, a final scan of the eye is generated by aligning the line scans relative to each other based upon the detected optical marker portion(s) of each line scan. The final line scan (which may thus be aligned to account for jitter, according to aspects described herein) may then be provided for display to a user, stored in a data store in association with an individual for which the retina scan was performed, or transmitted to a remote computing device, among other examples.

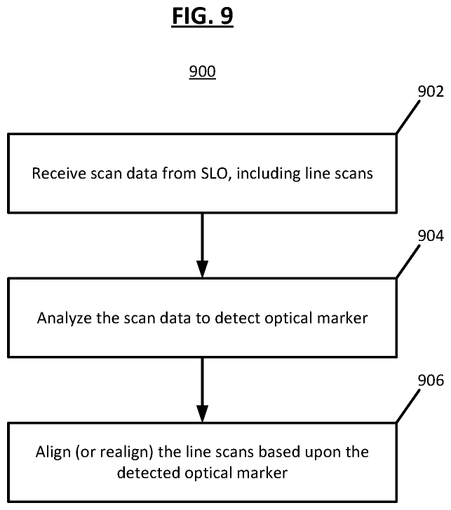

FIG. 9 shows a method 900 of scanning a retina of an eye using the SLO, and generating the final scan based on the optical marker for SLO imaging, according to examples disclosed herein. In step 902, the scan data is received from the SLO. The scan data may be generated, for example, using the method 800, such as via steps 802, 804, and 806. The scan data may include a plurality of line scans, each having an optical marker portion(s) corresponding to an optical marker(s) of an optical element of the SLO (e.g., the optical marker 214). In step 904, the scan data is analyzed to detect the optical marker portion(s) in the scan data. Specifically, the analysis may be performed to detect occurrences of the optical marker(s) that each thus show an alignment of a line scan relative to another line scan (e.g., an adjacent line scan or scans) of the plurality of line scans. As noted above, operation of step 904 may comprise filtering for an anomaly within the scan data that corresponds to a retinal feature of an individual rather than an optical marker, for example based on an expected period at which an optical marker is expected to be detected within the scan data and/or based on a color/pattern corresponding to the optical marker, among other examples.

In step 906, the plurality of line scans are aligned with each other based upon the detected optical marker portion(s), as explained herein. In instances where an optical element includes multiple optical markers, operation 906 may comprise scaling a given line scan according to multiple optical marker portions therein (e.g., in relation to corresponding optical marker portions of one or more adjacent line scans), thereby accounting for or otherwise reducing variability in line scan time, in addition to jitter as discussed above. Similar to step 810 discussed above, step 906 may additionally or subsequently comprise generating a final scan of the retina after detecting or confirming that all the line scans are aligned with each other, providing the final scan for display to a user, storing the final scan in association with an individual for which the retinal scan was performed, or transmitting the final scan to a remote computing device, among other examples. In some examples, step 906 may be performed directly following step 902 each time a new line scan of the scan data is generated, instead of in response to generating the scan data that includes the entirety of the line scans to be analyzed (e.g., step 904). That is, the aligning of the line scans in step 906 may be performed at or near real-time as new or additional line scans are obtained or generated in step 902. Whenever a new line scan is generated, the new line scan may be automatically aligned with the previously generated line scan (for example, the line scan that was last generated prior to generating the current, new line scan) based upon the optical marker portions that are included in the new line scan and the last generated line scan. Therefore, the adjustment or alignment of the line scans may be performed “on the fly” as each new line scan is generated, without having to wait for a predetermined number of line scans to be generated. In such examples, the final scan may be generated by simply combining the line scans that have already been adjusted or aligned with respect to each other into a single image.

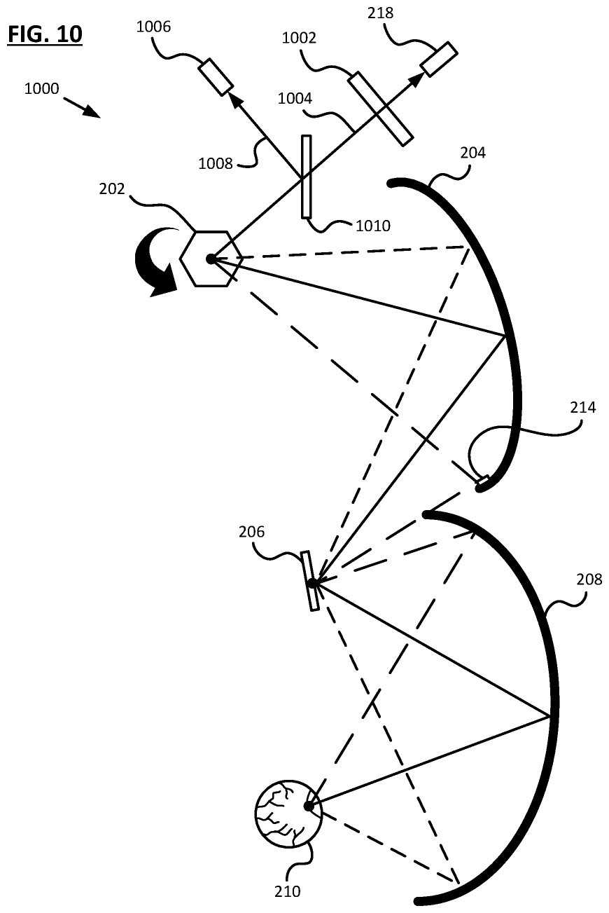

As shown in FIG. 10, the ophthalmic imaging system 100 may include or incorporate an autofluorescence (AF) imaging modality 1000 for the SLO 102 in which light that is reflected by a patient's eye 210 is filtered out using an optical filter 1002 such that only the fluorescent light is gathered by the detector 218 in a first channel 1004. For simplicity, the light source 200 is not shown in the figure. Beneficially, the filtering of light in such matter assists in imaging certain pathologies within the eye 210. However, in such modality, because the reflected light is filtered out, an optical marker 214 that implements only a bicolor barcode or a visible mark on the optical element may not be detected or scanned as efficiently (or, in some examples, only partially detected or scanned) due to the reflected light being filtered out before reaching the detector 218. In such examples, the optical marker 214 may be a fluorescent material that fluoresces at a predetermined wavelength or a predetermined range of wavelengths corresponding to the wavelength or range of wavelengths at which the material in the eye 210 will fluoresce.

In some examples, additionally or alternatively, a second channel 1008 may be implemented that uses a second or additional detector 1006 to acquire the reflected light before the reflected light is filtered out, where the second channel 1008 is different from the first channel 1004. In some examples, the reflected light may be redirected to the first channel 1004 and the second channel 1008 using a suitable component such as a beam splitter 1010, for example. The optical filter 1002 is thus located between the beam splitter 1010 and the detector 218, and reflected light is only filtered out in the first channel 1004. Thus, the second channel 1008 may enable detection of the optical marker 214, even though the optical marker 214 may be less visible or may not be detectable via the first channel 1004. In some examples, the AF imaging modality includes only one channel 1004 that is used to detect the fluorescent light, and, as such, the second channel 1008 may be used to gather the unfiltered reflected light. The unfiltered reflected light may thus be used to determine the position of the optical marker 214 in each line scan 602, and the determined movement or misalignment of line scans 602 may be used to correct the AF scan data (e.g., by processing optical marker portions in scan data from second detector 1006 in conjunction with AF scan data from detector 218).

In yet another example, two excitation lasers (generated by either the same light source in a collimated light beam, or generated by two different light sources and traveling parallel to each other) may be used simultaneously in the AF imaging modality 1000 such that one laser (first laser) is used to fluoresce the eye 210, and the other laser (second laser) uses a wavelength that either fluoresces the imaging target or simply is reflected by the imaging target, where the imaging target is the eye 210 or a portion/section thereof. In some examples, the two lasers may be combined or collimated into a single beam of light or a single scanning beam, which is then split into two separate individual lasers using a beam splitter, for example, before being separately received by two different detectors (e.g., first detector 218 and second detector 1006).

As such, the first detector 218 may be used to gather the fluorescent light from the patient's eye 210, where the second laser may be filtered out using an optical filter (e.g., optical filter 1002) before reaching the first detector 218. The second detector 1006 may be used to detect the optical marker 214, such that the position of the optical marker 214 in each line scan 602 may be determined as described above, and the resulting fluorescence scan data (generated based upon the filtered light as detected by the first detector 218) may then be corrected accordingly based on detected optical marker portions via the second detector 1006.

In some examples, the aforementioned issues and solutions may apply to fluorescein angiography (FA) and indocyanine green (ICG) angiography imaging modalities as well. In the FA and ICG modalities, a dye is injected into the bloodstream of the patient, and in some cases, the dye in the bloodstream of the patient may fluoresce and thus causing the aforementioned issues leading to possibly insufficient detection of the optical marker(s). As such, the aforementioned examples of addressing such issues also applies to the FA and ICG modalities.

FIG. 11 illustrates example physical components of a computing device 106, such as the computing device or devices associated with the ophthalmic imaging systems described above. As shown, the computing device 106 includes at least one processor such as CPU and/or GPU 1108, a system memory 1112, and a system bus 1110 that couples the system memory 1112 to the CPU/GPU 1108. The central processing unit 1108 is an example of a processing device.

The system memory 1112 includes a random access memory (“RAM”) 1118 and a read-only memory (“ROM”) 1120. A basic input/output (“I/O”) system containing the basic routines that help to transfer information between elements within the computing device, such as during startup, is stored in the ROM 1120. The computing device further includes a mass storage device 1114. The mass storage device 1114 is able to store software instructions and data. The mass storage device 1114 is connected to the CPU/GPU 1108 through a mass storage controller connected to the system bus 1110. The mass storage device 1114 and its associated computer-readable data storage media provide non-volatile, non-transitory storage for the computing device 106. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the device can read data and/or instructions. The mass storage device 1114 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device.

The computing device 106 may operate in a networked environment using logical connections to remote network devices through a network 1100, such as a local network, the Internet, or another type of network. The computing device 106 connects to the network 1100 through a network interface unit 1116 connected to the system bus 1110. The network interface unit 1116 may also connect to other types of networks and remote computing systems.

The computing device 106 includes an input/output controller 1122 for receiving and processing input from a number of other devices, including a touch user interface display screen, or another type of input device. Similarly, the input/output controller 1122 may provide output to a touch user interface display screen, a printer, or other type of output device.

As mentioned above, the mass storage device 1114 and the RAM 1118 of the computing device 106 can store software instructions and data. The software instructions include an operating system 1132 suitable for controlling the operation of the computing device 106. The mass storage device 1114 and/or the RAM 1118 also store software instructions, that when executed by the CPU/GPU 1108, cause the computing device 106 to provide the functionality discussed in this document, including the methods described herein and shown in the Figures.

Communication media may be embodied in the software instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, communication media may include wired media such as a wired network or direct-wired connection, and wireless media such as acoustic, radio frequency (RF), infrared, and other wireless media.

The block diagrams depicted herein are just examples. There may be many variations to these diagrams described therein without departing from the spirit of the disclosure. For instance, components may be added, deleted or modified.

In the foregoing description, example aspects are described with reference to several example embodiments. Accordingly, the specification is illustrative, rather than restrictive. Similarly, the figures illustrated in the drawings, which highlight the functionality and advantages of the example embodiments, are presented for example purposes only. The architecture of the example embodiments is sufficiently flexible and configurable, such that it may be utilized (and navigated) in ways other than those shown in the accompanying figures.

Software embodiments of the examples presented herein may be provided as, a computer program, or software, such as one or more programs having instructions or sequences of instructions, included or stored in an article of manufacture such as a machine-accessible or machine-readable medium, an instruction store, or computer-readable storage device, each of which can be non-transitory, in one example embodiment (and can form a memory or store). The program or instructions on the non-transitory machine-accessible medium, machine-readable medium, memory, instruction store, or computer-readable storage device or medium, may be used to program a computer system or other electronic device. The machine- or computer-readable device/medium, memory, instruction store, and storage device may include, but are not limited to, floppy diskettes, optical disks, and magneto-optical disks or other types of media/machine-readable medium/instruction store/storage device suitable for storing or transmitting electronic instructions. The techniques described herein are not limited to any particular software configuration. They may find applicability in any computing or processing environment. The terms “computer-readable medium”, “machine-accessible medium”, “machine-readable medium”, “memory”, “instruction store”, “computer-readable storage medium”, and “computer-readable storage device” used herein shall include any medium that is capable of storing, encoding, or transmitting instructions or a sequence of instructions for execution by the machine, computer, or computer processor and that causes the machine/computer/computer processor to perform any one of the methods described herein. Furthermore, it is common in the art to speak of software, in one form or another (e.g., program, procedure, process, application, module, unit, logic, and so on), as taking an action or causing a result. Such expressions are merely a shorthand way of stating that the execution of the software by a processing system causes the processor to perform an action to produce a result.

Some embodiments may also be implemented by the preparation of application-specific integrated circuits, field-programmable gate arrays, or by interconnecting an appropriate network of conventional component circuits.

Some embodiments include a computer program product. The computer program product may be a storage medium or media, memory, instruction store(s), or storage device(s), having instructions stored thereon or therein which can be used to control, or cause, a computer or computer processor to perform any of the procedures of the example embodiments described herein. The storage medium/memory/instruction store/storage device may include, by example and without limitation, an optical disc, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the computer-readable medium or media, memory, instruction store(s), or storage device(s), some implementations include software for controlling both the hardware of the system and for enabling the system or microprocessor to interact with a human user or other mechanism utilizing the results of the example embodiments described herein. Such software may include without limitation device drivers, operating systems, and user applications. Ultimately, such computer-readable media or storage device(s) further include software for performing example aspects of the invention, as described above.

Included in the programming and/or software of the system are software modules for implementing the procedures described herein. In some example embodiments herein, a module includes software, although in other example embodiments herein, a module includes hardware, or a combination of hardware and software.

While various example embodiments of the present invention have been described above, it should be understood that they have been presented by way of example, and not limitation. It will be apparent to persons skilled in the relevant art(s) that various changes in form and detail can be made therein. Thus, the present invention should not be limited by any of the above described example embodiments, but should be defined only in accordance with the following claims and their equivalents.

Further, the purpose of the Abstract is to enable the Patent Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that any procedures recited in the claims need not be performed in the order presented.

While this specification contains many specific embodiment details, these should not be construed as limitations on the scope of any inventions or of what may be claimed, but rather as descriptions of features specific to particular embodiments described herein. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A scanning laser ophthalmoscope (SLO) for scanning a retina of an eye, comprising:
a light source emitting a scanning beam;
a detector;
at least one optical element configured to direct the scanning beam;
at least one optical marker disposed at a predetermined region of the at least one optical element;
a first scanning element configured to direct the scanning beam between the light source and the at least one optical element; and
a second scanning element configured to direct the scanning beam with respect to the at least one optical element,
wherein the detector is configured to generate scan data of the retina of the eye comprising a plurality of line scans using the at least one optical element, and a portion of the at least one optical marker is included in each line scan of the plurality of line scans to indicate an alignment of the line scan relative to another line scan of the plurality of line scans.

2. The SLO of claim 1, further comprising a line start sensor disposed in an optical path of the scanning beam and configured to output a signal to the detector to record a new line scan each time the scanning beam is directed to the line start sensor by the first scanning element.

3. The SLO of claim 2, wherein the detector is operatively coupled with the light source.

4. The SLO of claim 1, wherein the plurality of line scans are generated by scanning the retina along a first direction using the first scanning element and positions of the plurality of line scans are varied along a second direction orthogonal to the first direction using the second scanning element.

5. The SLO of claim 1, further comprising an optical filter disposed between the detector and the first scanning element and configured to filter the scanning beam to be received by the detector.

6. The SLO of claim 5, wherein the optical filter is configured to filter a reflected light from the eye and allow a fluorescent light to pass through the optical filter and thereby be received by the detector.

7. The SLO of claim 6, further comprising a beam splitter and a second detector configured to receive the reflected light before being filtered out by the optical filter, thereby obtaining scan data via the second detector that includes one or more portions corresponding to the at least one optical marker.

8. The SLO of claim 1, wherein the at least one optical marker includes a plurality of optical markers, the plurality of optical markers including: a line-start optical marker defining a first end of the line scans, and a line-end optical marker defining a second end of the line scans.

9. An ophthalmic imaging system comprising:
the SLO of claim 1; and
one or more processing units and one or more memory units storing thereon instructions that, when executed by the one or more processing units, cause the one or more processing units to:
receive the scan data generated by the SLO;
detect a plurality of optical marker portions in the plurality of line scans of the scan data that each correspond to the optical marker; and
generate a final scan of the eye by aligning the plurality of line scans relative to each other based upon the detected optical marker portions.

10. An ophthalmic imaging system comprising:
the SLO of claim 1; and
one or more processing units and one or more memory units storing thereon instructions that, when executed by the one or more processing units, cause the one or more processing units to:
receive the scan data generated by the SLO, the scan data including an optical marker portion corresponding to the optical marker in a first line scan of the scan data; and
align the first line scan relative to a second line scan of the scan data based upon the detected optical marker portion.

11. The system of claim 10, wherein the first line scan is aligned relative to the second line scan at or near real-time, and the second line scan is from the scan data that is previously generated by the SLO.

12. The system of claim 9, further comprising a module for optical coherence tomography (OCT) imaging including optics for OCT imaging that are operatively coupled with the SLO.

13. The system of claim 12, wherein the optics for OCT are coupled with the SLO at a location between the light source and the first scanning element.

14. The system of claim 12, wherein the optics for OCT are coupled with the SLO at a location between the first scanning element and the at least one optical element.

15. The system of claim 12, wherein the optics for OCT are coupled with the SLO at the second scanning element.

16. The system of claim 12, wherein the SLO, the optics for OCT, and at least one of the one or more processing units are enclosed within a common housing.

17. A method for scanning a retina of an eye using a scanning laser ophthalmoscope (SLO), the method comprising:
emitting, by a light source, a scanning beam toward a first scanning element;
directing, by the first scanning element, the scanning beam between the light source and at least one optical element, the at least one optical element comprising at least one optical marker disposed at a predetermined region of the at least one optical element; and
generating, by a detector, scan data for the retina comprising a plurality of line scans captured via the second scanning element and a second scanning element using the at least one optical element, wherein a portion of the at least one optical marker is included in each line scan of the plurality of line scans to indicate an alignment of the line scan relative to another line scan of the plurality of line scans.

18. The method of claim 17, further comprising:
filtering, by an optical filter disposed between the detector and the first scanning element prior to generating the scan data, the scanning beam received by the detector.

19. The method of claim 18, wherein the optical filter is configured to filter a reflected light from the eye and allow a fluorescent light to pass through the optical filter and thereby be received by the detector.

20. The method of claim 19, further comprising receiving, by a second detector via a beam splitter, the reflected light before being filtered out by the optical filter, thereby obtaining scan data via the second detector that includes one or more portions corresponding to the optical marker.

21. The method of claim 17, wherein the directing the scanning beam between the light source and the at least one optical element comprises:
directing, by the first scanning element, the scanning beam over a first optical marker of the at least one optical marker that is disposed at a first predetermined region of the at least one optical element; and
directing, by the first scanning element, the scanning beam over a second optical marker of the at least one optical marker that is disposed at a second predetermined region of the at least one optical element that is different from the first predetermined region.

22. The method of claim 17, further comprising:
detecting a plurality of optical marker portions in the plurality of line scans of the scan data that each correspond to the optical marker; and
generating a final scan of the eye by aligning the plurality of line scans relative to each other based upon the detected optical marker portions.

23. The method of claim 17, further comprising:
aligning a first line scan of the scan data relative to a second line scan of the scan data based upon an optical marker portion included in the scan data, the optical marker portion corresponding to the at least one optical marker.

24. The method of claim 17, further comprising:
performing optical coherence tomography (OCT) in parallel with operating the SLO, such that an output of the SLO is used for tracking movement of the eye in performing the OCT.

25. A method of generating a scan of an retina of an eye, the method comprising:
receiving scan data from a scanning laser ophthalmoscope (SLO), the scan data comprising a plurality of line scans that each have an optical marker portion corresponding to an optical marker of at least one optical element of the SLO, such that the scan data includes a plurality of optical marker portions that each indicate an alignment of a line scan relative to another line scan of the plurality of line scans; and
aligning each line scan of the plurality of line scans based upon the optical marker portions.

26. The method of claim 25, further comprising:
analyzing, in response to receiving the scan data from the SLO, the scan data to detect the plurality of optical marker portions corresponding to the optical marker of the at least one optical element.

27. The method of claim 26, wherein:
the optical marker is a first optical marker;
the plurality of optical marker portions is a plurality of first optical marker portions; and
the method further comprising:
analyzing the scan data to detect a plurality of second optical marker portions corresponding to a second optical marker of the at least one optical element; and
analyzing the scan data further comprises scaling each line scan based on the plurality of second optical marker portions.
